(19) United States
(12) Patent Application Publication
(10) Pub. No.: US 2026/0096822 A1
(43) Pub. Date: Apr. 9, 2026

(54) CLIP DEVICE

(71) Applicant: OLYMPUS MEDICAL SYSTEMS CORP., Tokyo (JP)

(72) Inventor: Shogo Shindo, Hachioji-shi (JP)

(21) Appl. No.: 19/415,376

(22) Filed: Dec. 10, 2025

Related U.S. Application Data
(63) Continuation of application No. PCT/JP2024/021906, filed on Jun. 17, 2024.
(60) Provisional application No. 63/590,939, filed on Oct. 17, 2023.
(60) Provisional application No. 63/508,588, filed on Jun. 16, 2023.

(51) Int. Cl. A61B 17/128

(52) U.S. Cl. CPC A61B 17/1285

(57) ABSTRACT

This clip device comprises: a clip unit having arms that includes a first arm and a second arm, and a holder for supporting the arms so that the arms can be opened and closed; a sheath separably connected to the clip unit; and a regulating member capable of regulating an opening operation in which the first arm transitions from a closed state to an open state. The arms can transition to: a first form in which the first arm and the second arm can be opened and closed; and a second form in which the second arm can be opened and closed and the opening operation of the first arm is regulated by the regulating member.

### CROSS-REFERENCE TO RELATED APPLICATIONS

The present invention relates to a clip device. This application claims the benefit of U.S. Provisional Application No. 63/508,588, filed June 16, 2023, and U.S. Provisional Application No. 63/590,939, filed October 17, 2023, the entire contents of which are incorporated herein by reference.

### BACKGROUND OF THE INVENTION

### Field of the Invention

### Description of Related Art

In endoscopic treatment, medical devices such as clip devices for treating biological tissue are used. The medical devices are introduced to treatment sites by an applicator (an introduction device) that may be inserted through a channel of an endoscope.

United States Patent Application, Publication No. 2021-030425 describes a clipping device for treating biological tissue. In the clipping device described in United States Patent Application, Publication No. 2021-030425, a control wire for controlling a clip has a bent portion. Bending of the bent portion of the control wire causes the clip connected to the control wire to move significantly and come into contact with tissue.

### SUMMARY OF THE INVENTION

A clip device according to a first aspect of the present invention includes a clip unit including arms having a first arm and a second arm, and a holder configured to support the arm so that the arms are openable and closeable, a sheath separably connected to the clip unit, and a regulating member configured to regulate an opening operation in which the first arm transitions from a closed form to an open form, wherein the arms are transitioned between a first form in which the first arm and the second arm are openable and closeable, and a second form in which the second arm is openable and closeable and the opening operation of the first arm is regulated by the regulating member.

### BRIEF DESCRIPTION OF THE DRAWINGS

FIG. 1 is a view showing a clip device according to a first embodiment.

FIG. 2 is a cross-sectional view of a clip unit loaded into an applicator in the clip device.

FIG. 3 is a cross-sectional view of the clip unit loaded in the applicator.

FIG. 4 is a view showing the clip unit with a pair of arms in an open form.

FIG. 5 a view showing a clip locked in a closed form.

FIG. 6 a view showing the clip locked in the closed form.

FIG. 7 is a view showing the clip device in which a sheath and a holder are separated.

FIG. 8 is a view showing the clip device in which the sheath and the holder are separated.

FIG. 9 is a cross-sectional view of an operating part including a regulating member.

FIG. 10 is a view showing the pair of arms in a normal form (a first form).

FIG. 11 is a view showing a transition to a swing form (a second form).

FIG. 12 is a view showing the pair of arms in the second form.

FIG. 13 is a view showing the clip unit with the pair of arms swung.

FIG. 14 is a view showing a lock step using the clip unit.

FIG. 15 is a view showing a separation step using the clip unit.

FIG. 16 is a view showing a clip device according to a second embodiment.

FIG. 17 is a cross-sectional view of an operating part of the clip device.

FIG. 18 is a view showing a modified example of a regulating member in the clip device.

FIG. 19 is a view showing a clip device according to a third embodiment.

FIG. 20 is a view showing a regulating member in the clip device.

FIG. 21 is a view showing an operation of the regulating member.

FIG. 22 is a view showing a pair of arms in the second form.

FIG. 23 is a view showing a rotating member which is a modified example of a sliding member.

FIG. 24 is a view showing the rotating member.

FIG. 25 is a view showing a clip device according to a fourth embodiment.

FIG. 26 is a view showing a regulating member in the clip device.

FIG. 27 is a view showing the regulating member.

FIG. 28 is a view showing a pressing member which is a modified example of an inner sheath in the clip device.

FIG. 29 is a view showing an operation of the pressing member.

FIG. 30 is a view showing the operation of the pressing member.

FIG. 31 is a view showing a clip device according to a fifth embodiment.

FIG. 32 is a view showing a clip unit in the clip device.

### DETAILED DESCRIPTION OF THE INVENTION

### First embodiment

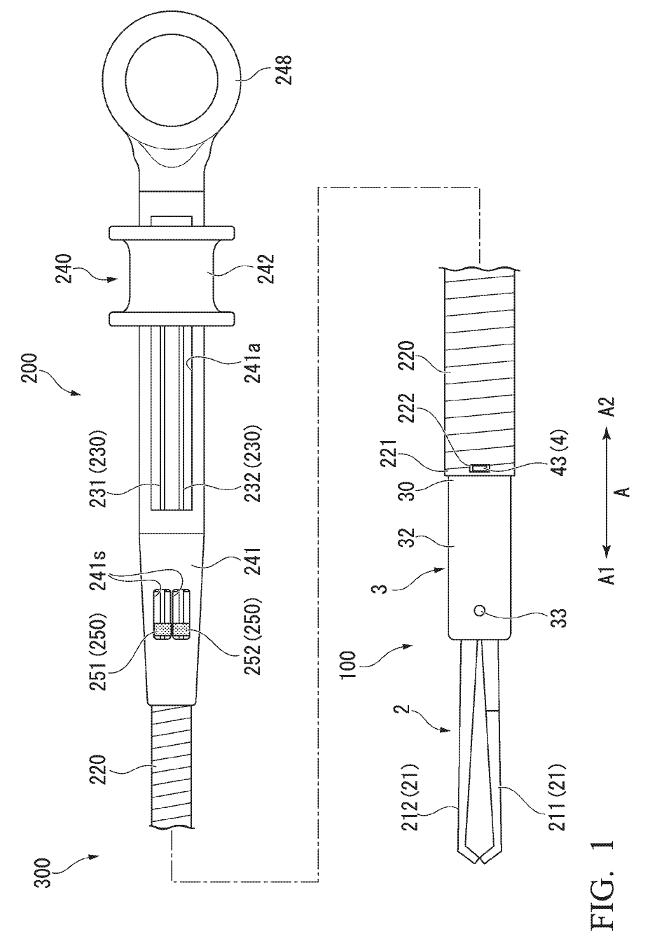

A first embodiment of the present invention will be described with reference to FIGS. 1 to 15.

FIG. 1 is a view showing a clip device 300 according to the embodiment.

The clip device (a clip system, an endoscopic treatment tool) 300 includes a clip unit 100 and an applicator 200. The clip unit 100 is loaded into the applicator 200 which may be inserted through a channel of an endoscope, and is introduced to a treatment position.

The clip device 300 may be capable of reloading the clip unit 100 into the applicator 200. In this case, a user may remove the used clip unit 100 from the applicator 200 and reload an unused clip unit 100 into the applicator 200.

In the following description, the clip unit 100 side in a longitudinal direction A of the clip device 300 will be referred to as the distal end side (the distal side) A1 of the clip device 300, and the operating part 240 side of the applicator 200 will be referred to as the proximal end side (the proximal side) A2 of the clip device 300.

### Applicator 200

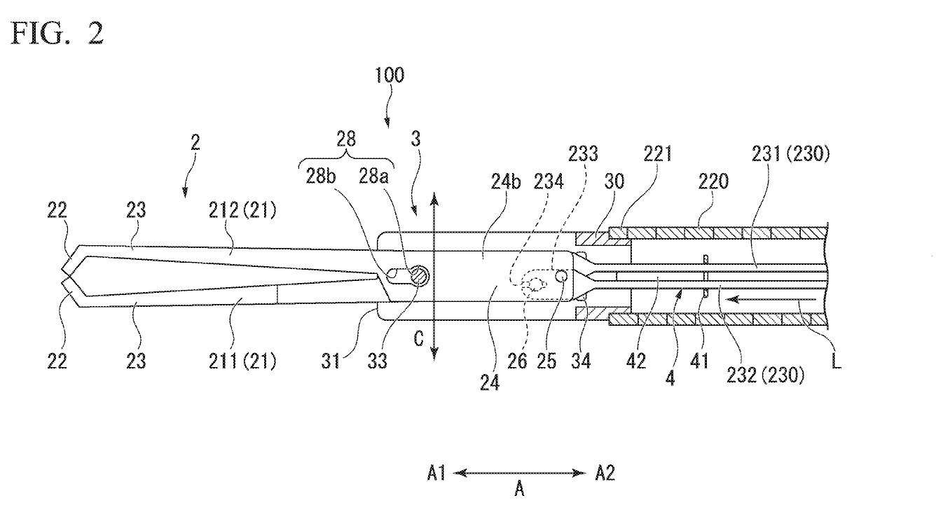

FIG. 2 is a cross-sectional view of the clip unit 100 loaded into the applicator 200.

The applicator (a clip unit introduction device) 200 includes a sheath 220, an operating wire 230, an operating part 240, and a regulating member 250. The applicator 200 is inserted, for example, through a treatment tool insertion channel of an endoscope and used in combination with the endoscope. Therefore, the sheath 220 is formed to be sufficiently longer than a length of the treatment tool insertion channel of the endoscope. The sheath 220 is flexible and curves in accordance with a curvature of an insertion part of the endoscope.

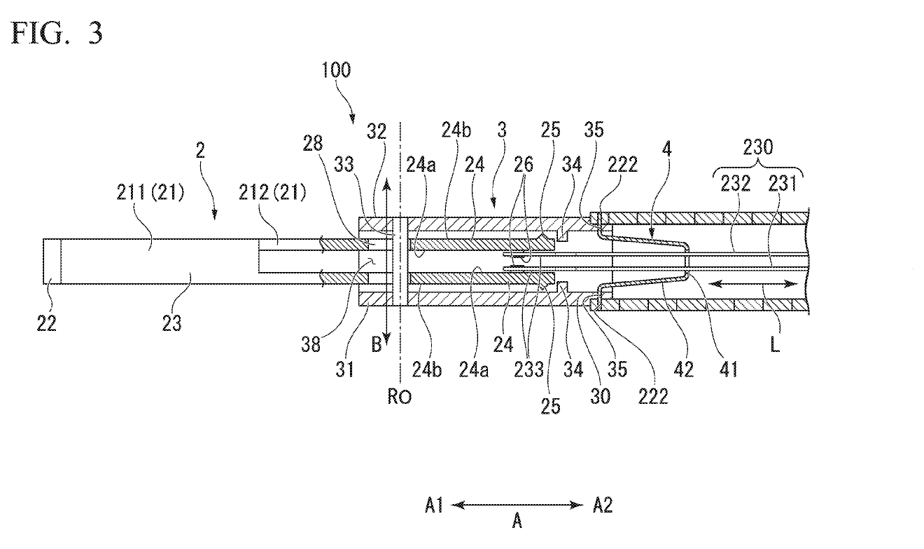

FIG. 3 is a cross-sectional view of the clip unit 100 loaded into the applicator 200.

The sheath 220 is a coil sheath. An engaging hole 222 that engages with a connecting member 4 of the clip unit 100 is provided in a distal end portion 221 of the sheath 220. The engaging hole 222 is provided on each of both sides of a central axis along a longitudinal axis direction L of the sheath 220.

The operating wire (a power transmission member) 230 is inserted through the sheath 220 and attached to the clip unit 100 and the operating part 240. As shown in FIG. 3, the operating wire 230 may protrude from the distal end portion 221 of the sheath 220 to the distal end side A1. The operating wire 230 advances and retracts through the distal end portion 221 along the longitudinal axis direction L of the sheath 220 to transmit power to a clip 2 of the clip unit 100.

A connecting portion 233 that is separably connected to a connecting portion 26 of the clip 2, which will be described below, is provided at the distal end of the operating wire 230. An outer diameter of the connecting portion 233 is larger than an outer diameter of the operating wire 230. A slit 234 into which the connecting portion 26 of the clip 2 which will be described below is fitted to be relatively movable is provided at the distal end of the connecting portion 233.

The operating wire 230 includes a first operating wire 231 and a second operating wire 232. The first operating wire 231 and the second operating wire 232 are arranged parallel to each other in an internal space of the sheath 220.

The operating part 240 includes an operating part main body 241, a slider 242, and a thumb ring 248. The operating part main body 241, the slider 242, and the thumb ring 248 are injection molded from, for example, a resin material. The operating part main body 241 has a slit portion 241a. The slit portion 241a supports the slider 242 so that the slider 242 may advance and retract.

The slider 242 is attached to the operating part main body 241 to be advanceable and retractable along the longitudinal direction A, and a proximal end of the operating wire 230 (the first operating wire 231 and the second operating wire 232) is attached thereto. When the slider 242 advances and retracts along the operating part main body 241, the operating wire 230 (the first operating wire 231 and the second operating wire 232) advances and retracts with respect to the sheath 220.

The thumb ring 248 is attached to a proximal end of the operating part main body 241 to be rotatable around a longitudinal axis of the operating part main body 241.

The regulating member 250 regulates an opening operation of the clip 2 of the clip unit 100, which transitions from a closed form (a closed state) to an open form (an open state). The regulating member 250 is provided at the operating part main body 241. The regulating member 250 has a first regulating member 251 that regulates movement of the first operating wire 231 to the distal end side A1, and a second regulating member 252 that regulates movement of the second operating wire 232 to the distal end side A1.

### Clip Unit 100

The clip unit 100 is loaded into the distal end portion 221 of the sheath 220 of the applicator 200. The clip unit 100 includes a clip 2, a holder 3, and a connecting member 4.

The clip (a clip arm) 2 has a pair of arms 21. The clip (the clip arm) 2 may have an auxiliary arm other than the pair of arms 21.

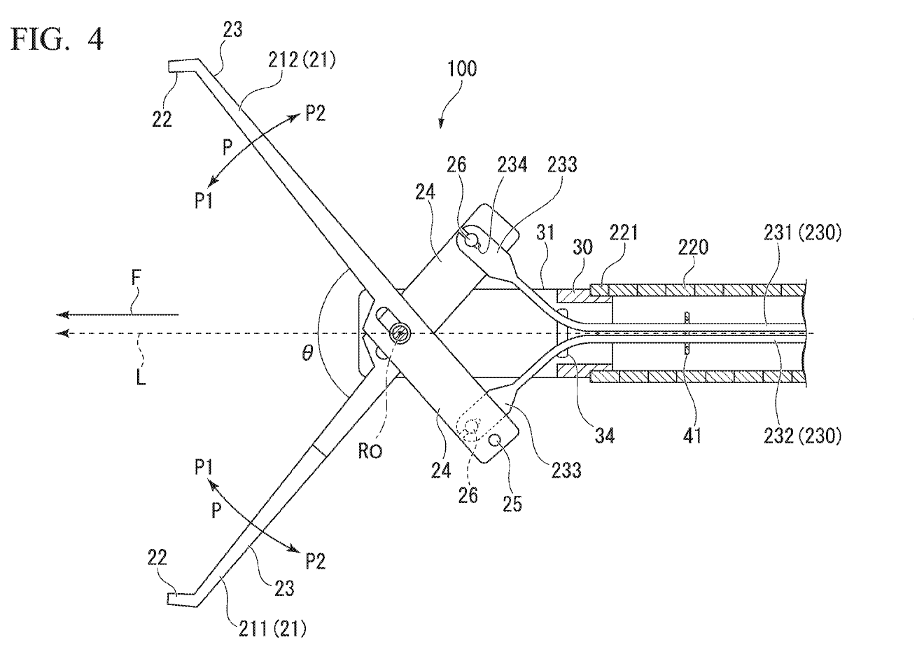

FIG. 4 shows the clip unit 100 with the pair of arms 21 in the open form.

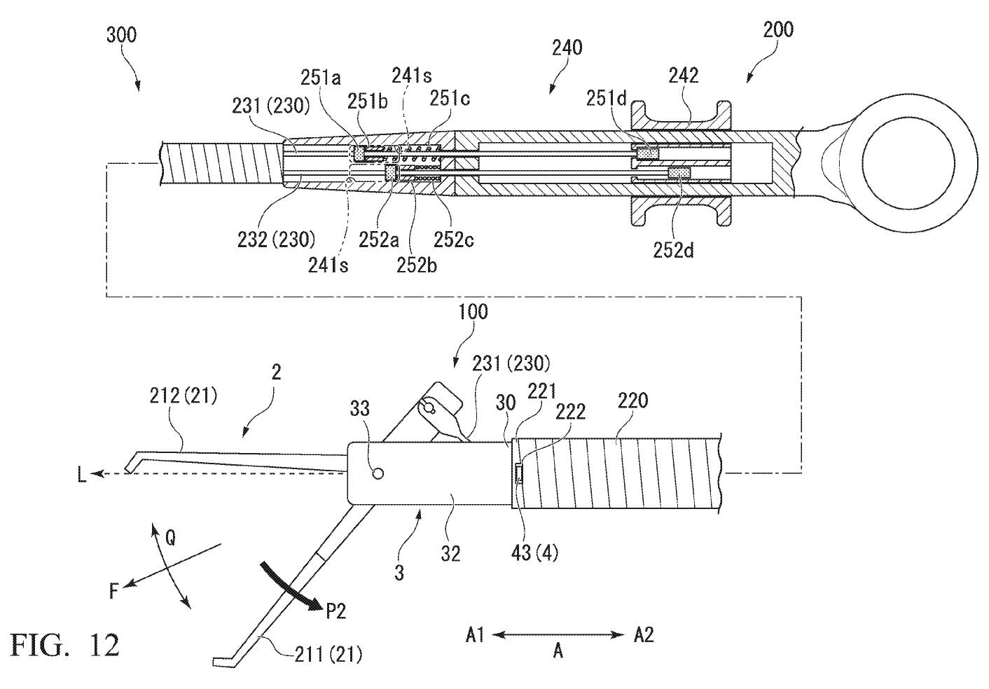

The pair of arms 21 may be opened and closed in a forward direction F, and may transition between the open form (the open state) and the closed form (the closed state). The pair of arms 21 may swing in a swing direction Q. When the pair of arms 21 swing, the forward direction F changes (refer to FIG. 12).

The pair of arms 21 includes a first arm 211 and a second arm 212. The first arm 211 and the second arm 212 are supported by the holder 3 to be rotatable in an opening-closing direction P around a rotation axis RO along an up-down direction B that is substantially perpendicular to the longitudinal direction A. The first arm 211 and the second arm 212 are separately supported by the holder 3.

In the opening-closing direction P, a direction in which one arm approaches the other arm is referred to as a “closing direction P1.” In the opening-closing direction P, a direction in which one arm moves away from the other arm is referred to as an “opening direction P2.” A side on which the pair of arms 21 face each other is referred to as the “inner side,” and the side opposite the inner side is referred to as the “outer side.” An operation of the pair of arms 21 transitioning from the open form (the open state) to the closed form (the closed state) is referred to as a “closing operation.” An operation of the pair of arms 21 transitioning from the closed form (the closed state) to the open form (the open state) is referred to as an “opening operation.”

Each of the first arm 211 and the second arm 212 has, from the distal end side A1 to the proximal end side A2, a claw 22, a gripping portion 23, a main body portion 24, and an engaging portion 25. The main body portion 24 has a connecting portion 26.

The claws 22 are formed by bending distal ends of the first arm 211 and the second arm 212 inward.

As shown in FIG. 2, the gripping portion 23 is formed in a flat plate shape and is a portion that grips tissue. The gripping portion 23 of the first arm 211 and the gripping portion 23 of the second arm 212 are provided so that flat plate surfaces thereof face each other.

As shown in FIG. 3, the main body portion 24 is formed in a flat plate shape and is supported by a pin 33 of the holder 3 to be rotatable in the opening-closing direction P. Specifically, a through hole 28 formed in an inner surface 24a of the main body portion 24 is fitted onto the pin 33 of the holder 3, whereby the main body portion 24 is supported rotatably with respect to the holder 3. The main body portion 24 is provided so that a thickness direction of the main body portion 24 substantially coincides with the up-down direction B.

The engaging portion 25 is a portion that may engage with an engaging portion 34 of the holder 3. The engaging portion 25 is a convex portion that protrudes outward from an outer surface 24b of the main body portion 24.

As shown in FIG. 2, the through hole 28 has a first through hole 28a that is circular when seen in the up-down direction B, and a second through hole 28b that is oval when seen in the up-down direction B. The second through hole 28b is connected to the first through hole 28a and extends from the first through hole 28a to the distal end side A1.

The connecting portion 26 is a portion that is separably connected to the slit 234 of the connecting portion 233 of the operating wire 230. The connecting portion 26 is a convex portion that protrudes inward from the inner surface 24a of the main body portion 24. When the operating wire 230 is pulled with a force equal to or greater than a predetermined value, the connecting portion 26 comes out of the slit 234, and the operating wire 230 and the connecting portion 26 are separated.

The operating wire 230 moves to the distal end side A1 so that the pair of arms 21 perform the opening operation. The operating wire 230 moves to the proximal end side A2 so that the pair of arms 21 perform the closing operation. An advancing and retracting direction of the operating wire 230 that causes the pair of arms 21 to perform the opening operation is referred to as an “opening direction.” The advancing and retracting direction of the operating wire 230 that causes the pair of arms 21 to perform the closing operation is referred to as a “closing direction.” In the embodiment, the opening direction is a direction in which the operating wire 230 is moved to the distal end side A1, and the closing direction is a direction in which the operating wire 230 is moved to the proximal end side A2. The opening direction may be a direction in which the operating wire 230 is moved to the proximal end side A2, and the closing direction may be a direction in which the operating wire 230 is moved to the distal end side A1.

The holder 3 supports the clip 2 to be rotatable in the opening-closing direction P around the rotation axis RO along the up-down direction B that is substantially perpendicular to the longitudinal direction A. The holder 3 has an internal space 38 in which the main body portion 24 of the clip 2 is stored.

As shown in FIG. 3, the holder 3 has a proximal end tubular portion 30, a first support portion 31, a second support portion 32, the pin 33, and the engaging portion 34.

The proximal end tubular portion 30 is a hard member formed of a metal or the like, and is formed in an annular shape. An engaging hole 35 with which the connecting member 4 engages is provided in the proximal end tubular portion 30. The engaging hole 35 is provided on each of both sides of a central axis along the longitudinal axis direction L of the sheath 220.

Each of the first support portion 31 and the second support portion 32 is formed in a plate shape and extends from the proximal end tubular portion 30 to the distal side A1. The first support portion 31 and the second support portion 32 are provided to face each other in the up-down direction B. The pair of arms 21 are disposed between the first support portion 31 and the second support portion 32.

The pin 33 is fixed to the first support portion 31 and the second support portion 32. The pin 33 passes through the through holes 28 of the main body portions 24 of the pair of arms 21. The pin 33 is formed in a cylindrical shape. A central axis of the pin 33 is the rotation axis RO of the pair of arms 21.

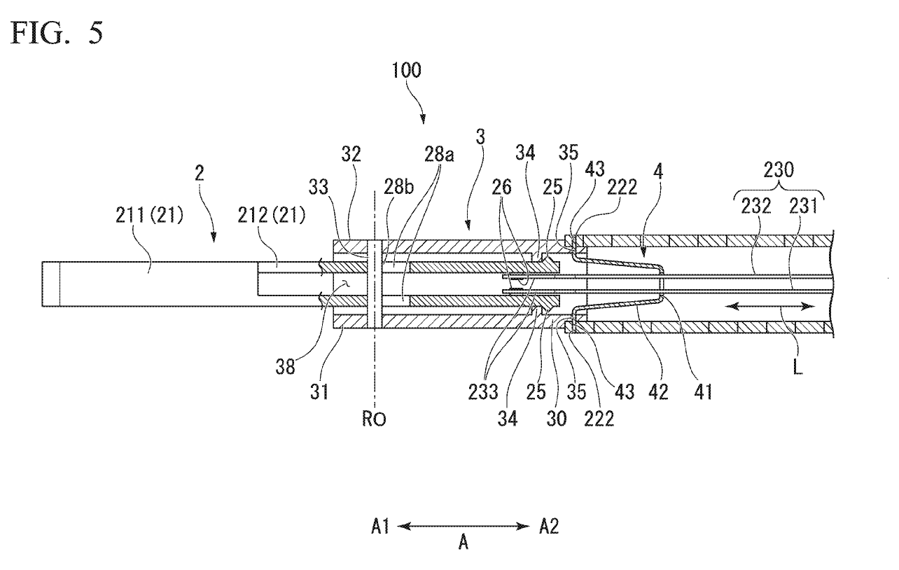

FIG. 5 is a view showing the clip 2 locked in the closed form.

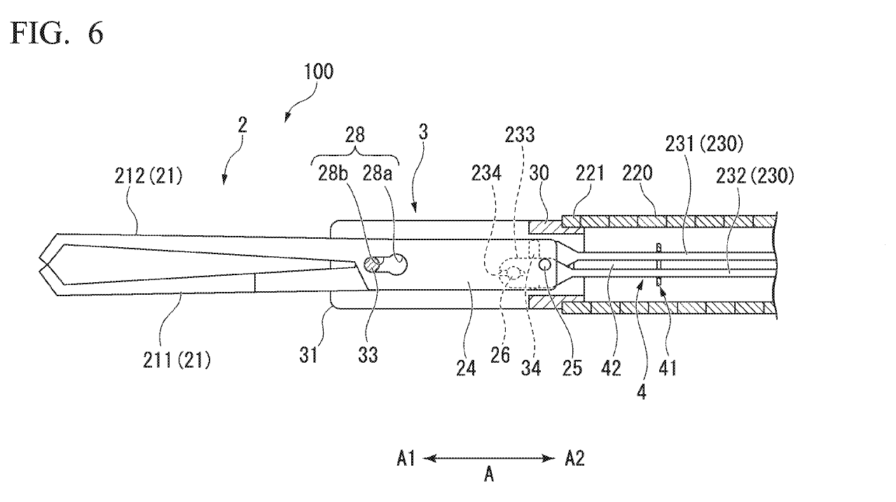

The engaging portion 34 is a portion that may engage with the engaging portion 25 of each of the pair of arms 21. The engaging portion 34 is a convex portion that protrudes from an inner circumferential surface of the proximal end tubular portion 30 toward the pair of arms 21. As shown in FIG. 6, when the pair of arms 21 move to the proximal end side A2 and the engaging portion 25 moves further to the proximal end side A2 than the engaging portion 34, the engaging portion 25 may no longer move further to the distal end side A1 than the engaging portion 34.

FIG. 6 shows the clip 2 locked in the closed form.

When the clip 2 moves to the proximal end side A2 so that a position of the pin 33 moves from the first through hole 28a to the second through hole 28b, the engaging portion 25 moves further to the proximal end side A2 than the engaging portion 34. By pulling the pair of clips 2 to the proximal end side A2, the pair of clips 2 transition to the closed form, and the position of the pin 33 moves from the first through hole 28a to the second through hole 28b. As a result, the engaging portion 25 moves further to the proximal end side A2 than the engaging portion 34, and the pair of clips 2 are locked in the closed form.

The engaging portion 25 and the engaging portion 34 of the holder 3 constitute a locking mechanism that locks the clip 2 in the closed form. Once the pair of arms 21 are locked in the closed form by the locking mechanism, the pair of arms 21 may not return to the open form. The locking mechanism is not limited to a combination of the engaging portion 25 of the clip 2 and the engaging portion 34 of the holder 3, and may have other configurations.

The connecting member 4 separably connects the sheath 220 and the holder 3. The connecting member 4 has an annular portion 41 and a pair of support arms 42.

The annular portion 41 is formed in a ring shape. The operating wires 230 (the first operating wire 231 and the second operating wire 232) are inserted through the annular portion 41.

The pair of support arms 42 have elasticity and are provided on both sides of a central axis along the longitudinal axis direction L of the sheath 220. Distal end portions 43 of the pair of support arms 42 are inserted through the engaging hole 222 of the sheath 220 and the engaging hole 35 of the holder 3. The distal end portions 43 are inserted through the engaging hole 222 of the sheath 220 and the engaging hole 35 of the holder 3 to connect the sheath 220 and the holder 3.

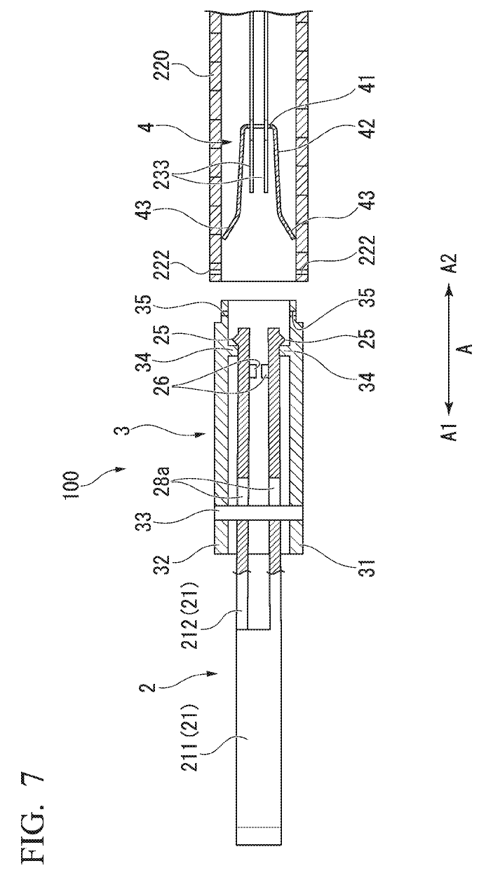

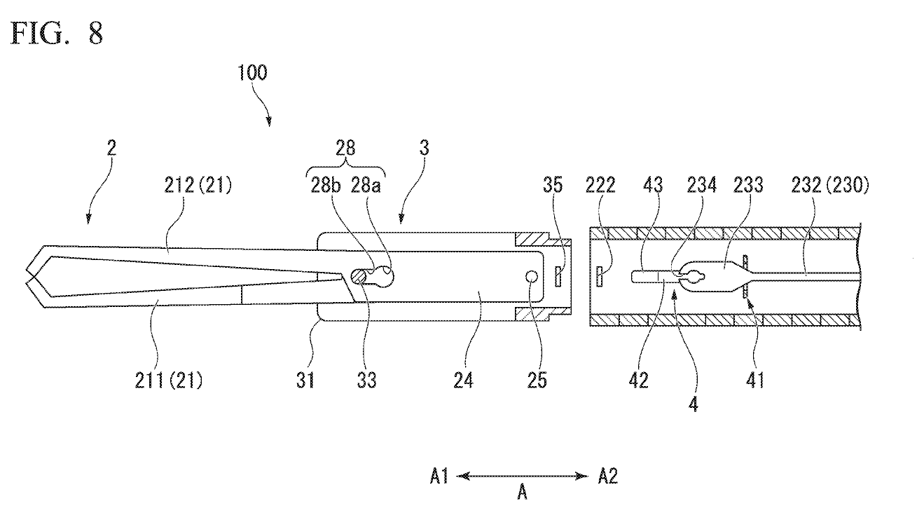

FIGS. 7 and 8 are views showing the clip device 300 in which the sheath 220 and the holder 3 are separated. When the clip 2 is locked in the closed form, and then the operating wire 230 is further pulled to the proximal end side A2, the slit 234 of the connecting portion 233 is disengaged from the connecting portion 26 of the clip 2. When the operating wire 230 is further pulled to the proximal end side A2, the connecting portion 233 engages with the annular portion 41 and moves the connecting member 4 to the proximal end side A2. The distal end portion 43 of the connecting member 4 is disengaged from the engaging hole 222 of the sheath 220 and the engaging hole 35 of the holder 3. As a result, the sheath 220 and the holder 3 are separated.

### Operation and Action of Regulating Member 250

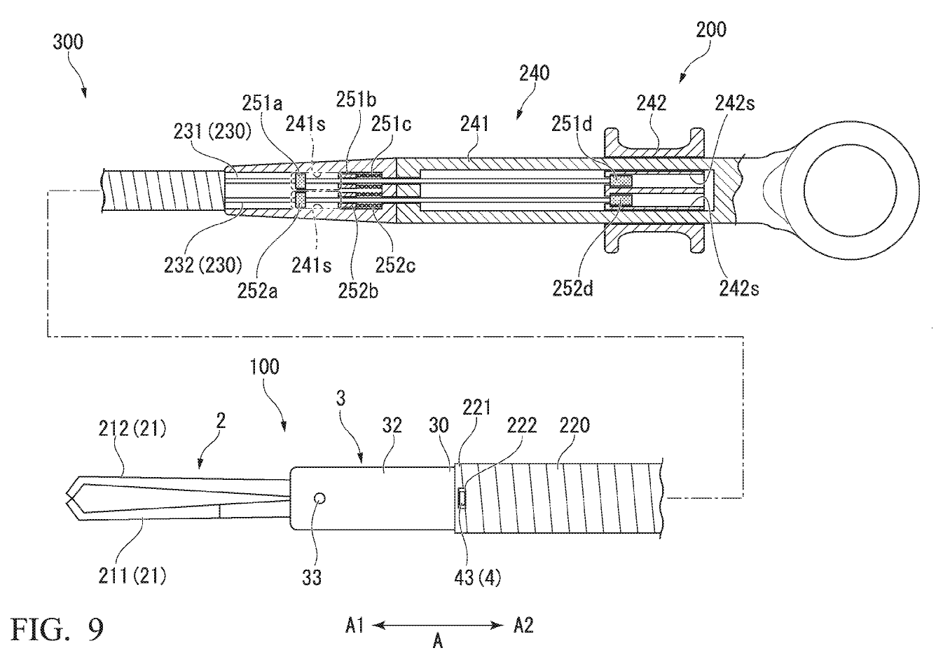

Next, an operation and action of regulating member 250 will be described with reference to FIGS. 9 to 12.

FIG. 9 is a cross-sectional view of the operating part 240 including the regulating member 250.

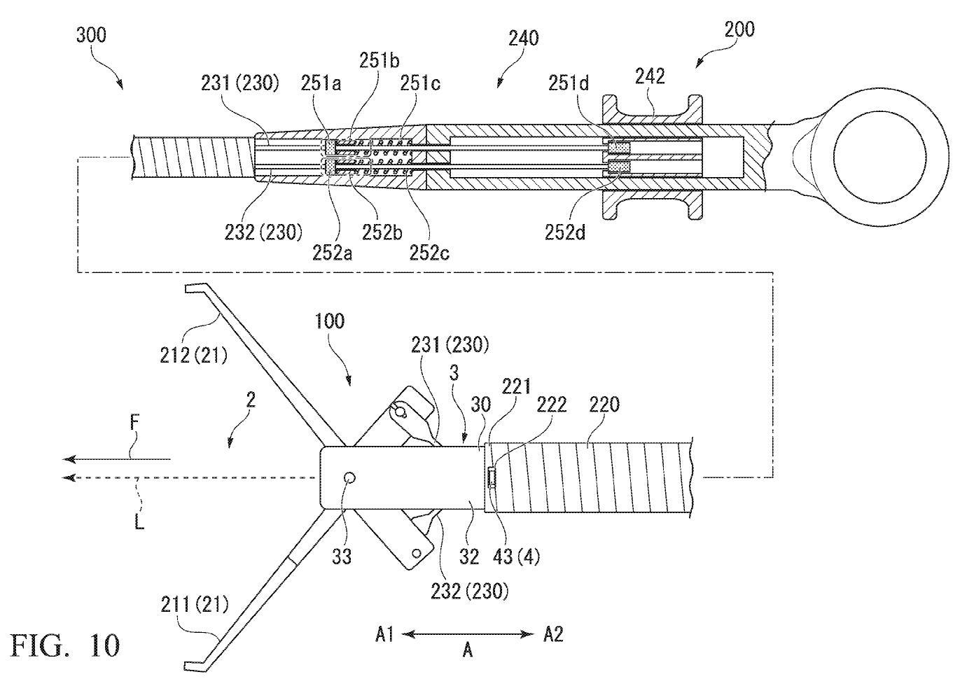

The regulating member 250 transitions the pair of arms 21 between a normal form (a first form, FIG. 10) and a swing form (a second form, FIG. 12).

The regulating member 250 includes a first regulating member 251 that regulates movement of the first operating wire 231 to the distal end side A1 (the opening direction), and a second regulating member 252 that regulates movement of the second operating wire 232 to the distal end side A1 (the opening direction).

The first regulating member 251 includes a first regulating knob 251a, a first knob engaging portion 251b, a first spring 251c, and a first slider engaging portion 251d.

The first control knob 251a is a knob attached to be advanceable and retractable in a slit 241s formed in the operating part main body 241 along the longitudinal direction A. An operator may move the first control knob 251a between a position on the distal end side A1 and a position on the proximal end side A2.

The first knob engaging portion 251b is fixed to a predetermined position on the first operating wire 231. The first knob engaging portion 251b is fixed to a position at which it may engage with the first regulating knob 251a disposed at the position on the proximal end side A2.

The first spring 251c is a compression spring. The first operating wire 231 is inserted through the first spring 251c. A distal end of the first spring 251c is attached to the first knob engaging portion 251b. A proximal end of the first spring 251c is attached to the operating part main body 241. The first spring 251c pushes the first knob engaging portion 251b and the first operating wire 231 to the distal end side A1. The first regulating member 251 does not necessarily have to have the first spring 251c.

The first slider engaging portion 251d is provided at the proximal end of the first operating wire 231, and is connected to the slider 242. The first slider engaging portion 251d is attached to be advanceable and retractable in a slit 242s formed in the slider 242 along the longitudinal direction A. The first slider engaging portion 251d advances and retracts in conjunction with the slider 242 until a predetermined force is applied in the longitudinal direction A. The first slider engaging portion 251d advances and retracts with respect to the slider 242 when a force equal to or greater than a predetermined value is applied in the longitudinal direction A. The first slider engaging portion 251d engages with an end portion of the slit 242s in the longitudinal direction A, and thus may not move outside the slit 242s.

The second regulating member 252 includes a second regulating knob 252a, a second knob engaging portion 252b, a second spring 252c, and a second slider engaging portion 252d. The second regulating member 252 is the same as the first regulating member 251 except that the second regulating member 252 regulates the movement of the second operating wire 232.

The regulating member 250 may have at least one of the first regulating member 251 and the second regulating member 252. For example, when the regulating member 250 has the first regulating member 251 but does not have the second regulating member 252, the regulating member 250 regulates the movement of only the first operating wire 231.

As shown in FIG. 9, by moving the first regulating knob 251a and the second knob engaging portion 252b to the distal end of the slit 241s, the pair of arms 21 transitions to the normal form (the first form).

FIG. 10 shows the pair of arms 21 in the normal form (the first form).

The normal form (the first form) is an operation form in which the first arm 211 and the second arm 212 may be opened and closed. In the embodiment, the first arm 211 and the second arm 212 may be opened and closed in coordination with each other. As shown in FIG. 10, when the operator moves the slider 242 to the distal end side A1, the regulating member 250 does not regulate the movement of the operating wire 230, and the slider 242 advances the first operating wire 231 and the second operating wire 232 in coordination. The forward direction F of the pair of arms 21 is along the longitudinal axis direction L of the sheath 220. When the pair of arms 21 are in the normal form, the forward direction F of the pair of arms 21 does not necessarily have to be along the longitudinal axis direction L of the sheath 220.

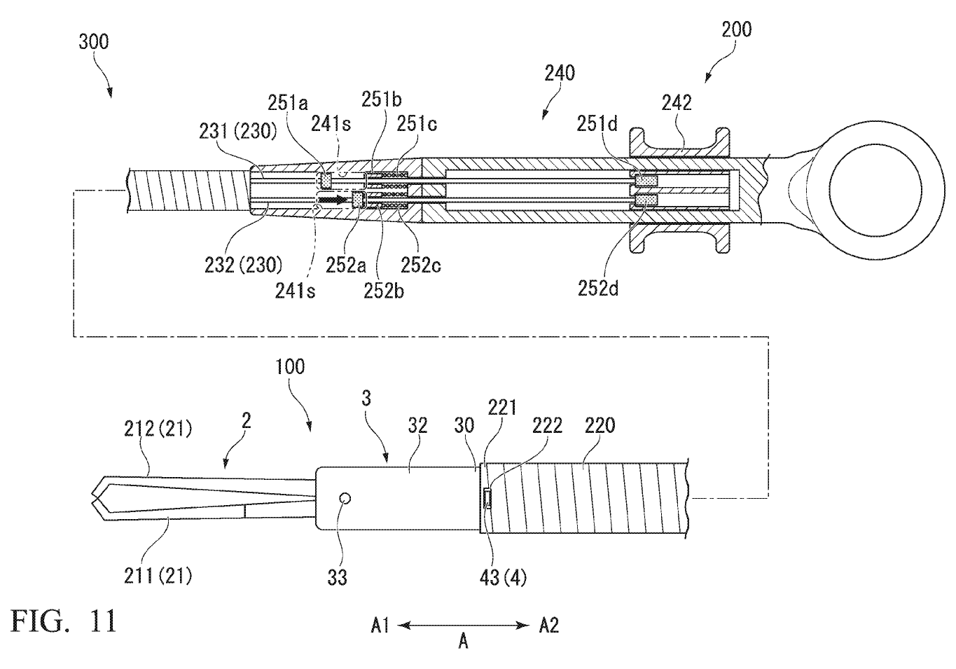

FIG. 11 is a view showing transition to the swing form (the second form).

By moving one of the first regulating knob 251a and the second knob engaging portion 252b to the proximal end of the slit 241s, the pair of arms 21 transition to the swing form (the second form).

FIG. 12 shows the pair of arms 21 in the second form.

The swing form (the second form) is an operation form in which one of the first arm 211 and the second arm 212 may be opened and closed, and an opening operation of the other is regulated by the regulating member 250. The first regulating knob 251a that has moved to the proximal end regulates the movement of the first operating wire 231 to the distal end side A1 (in the opening direction), thereby regulating the opening operation of the first arm 211. Alternatively, the second regulating knob 252a that has moved to the proximal end regulates the movement of the second operating wire 232 to the distal end side A1 (in the opening direction), thereby regulating the opening operation of the second arm 212. As shown in FIG. 12, when the operator moves the slider 242 to the distal end side A1 (in the opening direction), the regulating member 250 regulates the movement of the second operating wire 232 to the distal end side A1 (in the opening direction), and the slider 242 moves only the first operating wire 231 to the distal end side A1 (in the opening direction). As a result, only the first arm 211 is in the open form. The forward direction F of the pair of arms 21 is along a direction intersecting the longitudinal axis direction L of the sheath 220.

As shown in FIG. 12, the movement of the second operating wire 232 to the distal end side A1 (in the opening direction) is regulated. Therefore, a force equal to or greater than a predetermined value is applied to the second slider engaging portion 252d in the longitudinal direction A, and the second slider engaging portion 252d moves to the proximal end side A2 with respect to the slider 242.

### Operation and Action of Clip Device 300

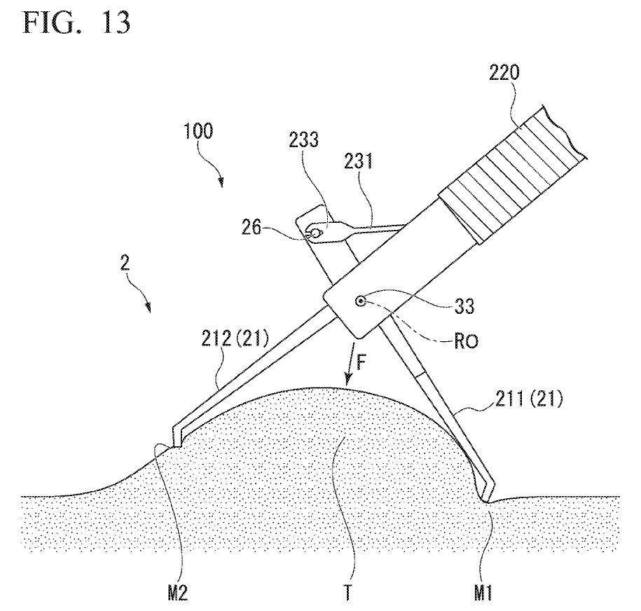

Next, an operation and action of the clip device 300 will be described with reference to FIGS. 13 to 15.

### Approach Step

The operator introduces the clip unit 100 loaded into the applicator 200 into the body via an endoscope channel. The operator moves the endoscope and the sheath 220 to bring the clip 2 of the clip unit 100 closer to tissue T which is a treatment region.

A method for causing the clip unit 100 to approach the treatment region includes at least a frontal approach and a tangential approach. In the frontal approach, the operator operates the endoscope and the sheath 220 to arrange the clip unit 100 so that the longitudinal direction A of the clip unit 100 is approximately perpendicular to a surface of the treatment region. In the tangential approach, the operator operates the endoscope and the sheath 220 to arrange the clip unit 100 so that the longitudinal direction A of the clip unit 100 is along the surface of the treatment region.

### Tangential Approach

FIG. 13 shows the clip unit 100 with the pair of arms 21 swung.

The operator uses the tangential approach to bring the clip 2 closer to the tissue T which is the treatment region. The operator operates the second regulating member 252 to regulate the opening operation of the second arm 212 which hooks onto a mucous membrane (a second mucous membrane M2 in FIG. 13) located at the distal end side (the inner side, the far side) of a first mucous membrane M1 and the second mucous membrane M2 on both side of a center of the tissue T. Specifically, the operator regulates the opening operation of the second arm 212 by moving the second knob engaging portion 252b to the proximal end of the slit 241s. The operator moves the slider 242 to the distal end side A1 so that only the first arm 211 is in the open form. The pair of arms 21 transition to the swing form (the second form). The pair of arms 21 is disposed so that the forward direction F faces a front surface of the tissue T which is the treatment region. The operator grips the first mucous membrane M1 and the second mucous membrane M2 with the pair of arms 21.

### Pulling Step

The user retracts the operating wire 230 by retracting the slider 242 along the operating part main body 241. The connecting member 4 connected to the operating wire 230 pulls the clip 2. As the clip 2 is pulled to the proximal end side A2, the pair of arms 21 (the first arm 211 in FIG. 13) gradually close.

### Lock Step

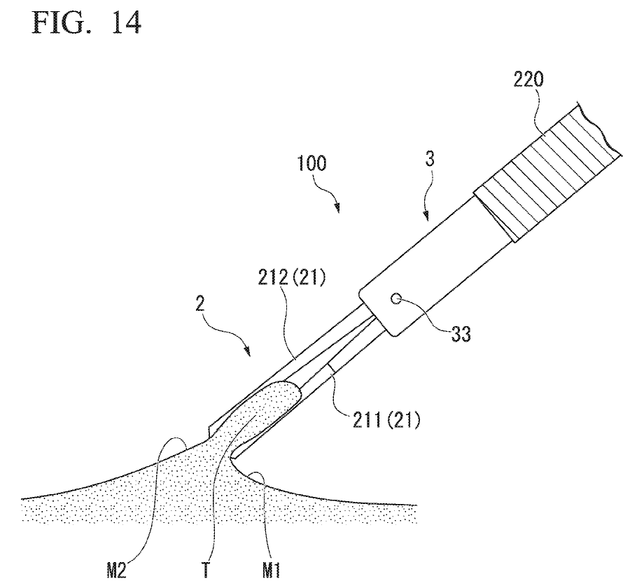

FIG. 14 shows a lock step performed by the clip unit 100.

The operator further retracts the operating wire 230 by retracting the slider 242 along the operating part main body 241. When the clip 2 is pulled to a predetermined position in the holder 3, the engaging portions 25 of the clip 2 engage with the engaging portion 34 of the holder 3, and the pair of arms 21 are locked in the closed state. Once the pair of arms 21 are locked in the closed state, the pair of arms 21 may not return to the open state.

### Separation Step

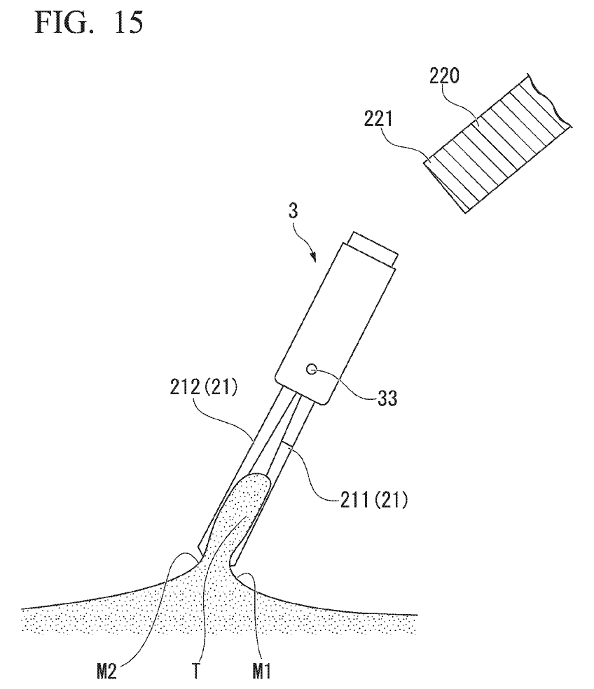

FIG. 15 shows a separation step using the clip unit 100.

The operator further pulls the clip 2. The distal end portion 43 of the connecting member 4 disengages from the engaging hole 222 of the sheath 220 and the engaging hole 35 of the holder 3. As a result, the sheath 220 and the holder 3 are separated. The operator retracts the sheath 220, and detains the clip 2 and holder 3 in the body in which the first mucous membrane M1 and the second mucous membrane M2 are ligated.

The clip device 300 according to the embodiment can suitably grip the tissue T in a lumen. Even when the tissue T is treated using the tangential approach, the clip 2 may be swung so that the pair of arms 21 are disposed to face the front surface of the tissue T which is the treatment region. In the clip device 300, since the clip 2 is swung by regulating the opening operation of one of the arms 21, the pair of arms 21 may be easily disposed to face the front surface of the tissue T even in a narrow lumen.

Although the first embodiment of the present invention has been described above in detail with reference to the drawings, the specific configuration is not limited to the embodiment, and design changes and the like are also included within the scope that does not deviate from the gist of the present invention. Furthermore, the components shown in the embodiments and modified examples can be appropriately combined to form a configuration.

### Second embodiment

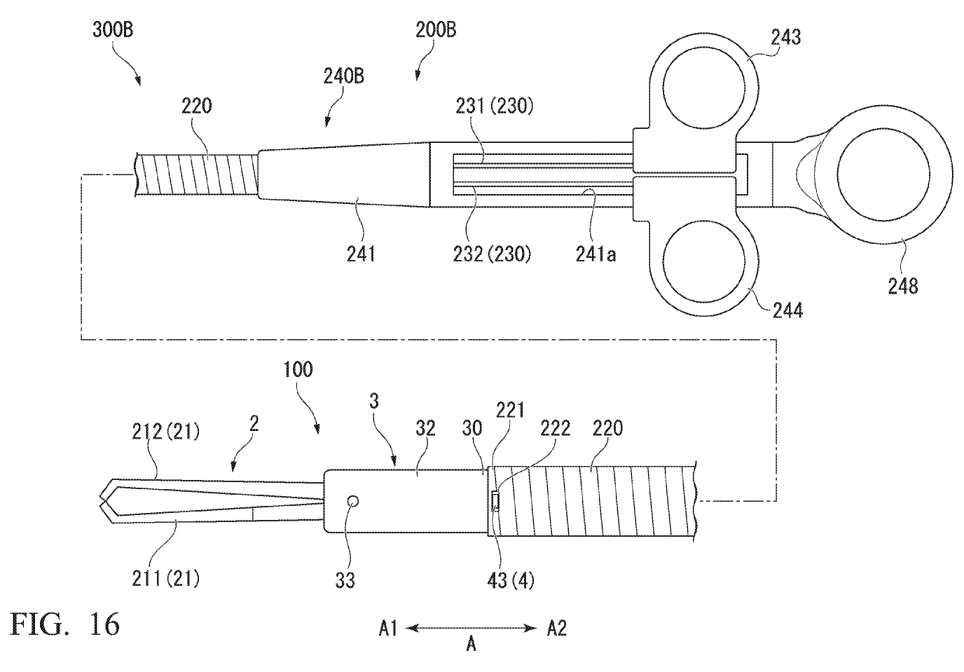

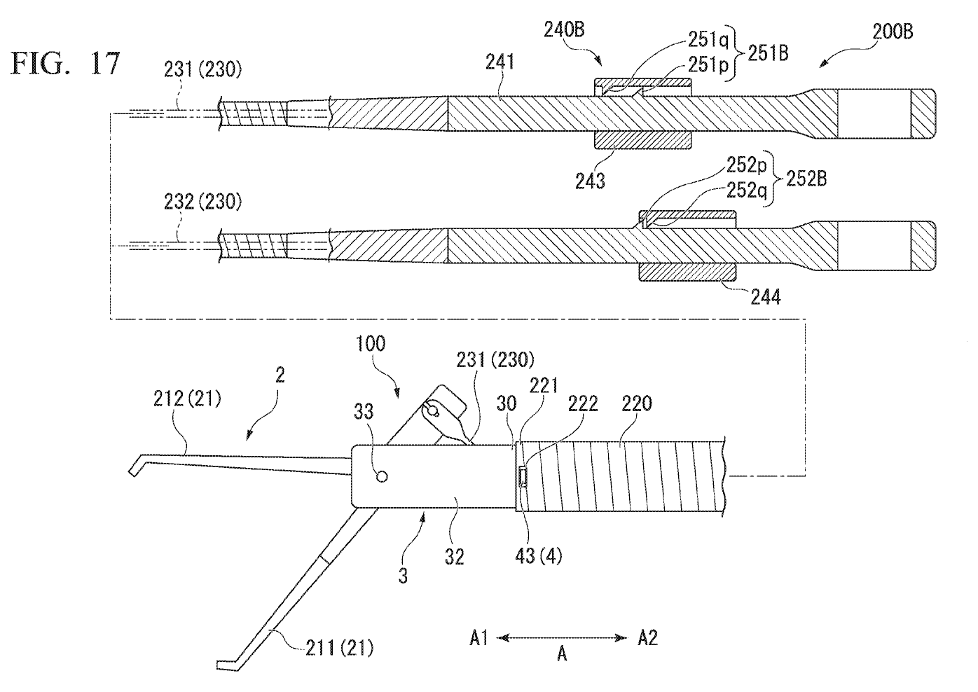

A clip device 300B according to a second embodiment of the present invention will be described with reference to FIGS. 16 and 17. In the following description, components that are common to those already described will be assigned the same reference numerals, and duplicate descriptions will be omitted.

FIG. 16 shows the clip device 300B according to the embodiment.

The clip device (a clip system, an endoscopic treatment tool) 300B includes a clip unit 100 and an applicator 200B. The clip unit 100 is loaded into the applicator 200B that may be inserted through a channel of an endoscope, and is introduced into a treatment position.

The applicator (a clip unit introduction device) 200B includes a sheath 220, an operating wire 230, an operating part 240B, and a regulating member 250B.

The operating part 240B includes an operating part main body 241, a first slider 243, a second slider 244, and a thumb ring 248.

The first slider 243 is attached to the operating part main body 241 to be advanceable and retractable in the longitudinal direction A, and is attached to a proximal end of the first operating wire 231. When the first slider 243 advances and retracts along the operating part main body 241, the first operating wire 231 advances and retracts with respect to the sheath 220.

The second slider 244 is attached to the operating part main body 241 to be advanceable and retractable in the longitudinal direction A, and is attached to a proximal end of the second operating wire 232. When the second slider 244 advances and retracts along the operating part main body 241, the second operating wire 232 advances and retracts with respect to the sheath 220.

FIG. 17 is a cross-sectional view of the operating part 240B.

The regulating member 250B transitions the pair of arms 21 between the normal form (the first form) and the swing form (the second form).

The regulating member 250B includes a first regulating member 251B that regulates the movement of the first operating wire 231 to the distal end side A1 (the opening direction), and a second regulating member 252B that regulates the movement of the second operating wire 232 to the distal end side A1 (the opening direction). The regulating member 250B may have only one of the first regulating member 251B and the second regulating member 252B, and may not have the other.

The first regulating member 251B is a ratchet that regulates an operation of the first slider 243 that moves the first operating wire 231 in the opening direction. Specifically, the first regulating member 251B includes a main body first convex portion 251p provided on the operating part main body 241 and a first slider convex portion 251q provided on the first slider 243. The first slider convex portion 251q easily climbs over the main body first convex portion 251p to the proximal end side A2 (the closing direction), but has difficulty in climbing over the main body first convex portion 251p to the distal end side A1 (the opening direction).

The second regulating member 252B is a ratchet that regulates an operation of the second slider 244 that moves the second operating wire 232 in the opening direction. Specifically, the second regulating member 252B includes a main body second convex portion 252p provided on the operating part main body 241 and a second slider convex portion 252q provided on the second slider 244. The second slider convex portion 252q easily climbs over the main body second convex portion 252p to the proximal end side A2 (the closing direction), but has difficulty in climbing over the main body second convex portion 252p to the distal end side A1 (the opening direction).

By further moving the first slider convex portion 251q to the distal end side A1 (the opening direction) than the main body first convex portion 251p and further moving the second slider convex portion 252q to the distal end side A1 (the opening direction) than the main body second convex portion 252p, the pair of arms 21 transition to the normal form (the first form).

The normal form (the first form) is an operation form in which the first arm 211 and the second arm 212 may be opened and closed. In the embodiment, the first arm 211 and the second arm 212 may be opened and closed independently. At this time, the first regulating member 251B does not regulate the advance and retract movement of the first operating wire 231. The second regulating member 252B does not regulate the advance and retract movement of the second operating wire 232.

By further moving one of the first slider convex portion 251q and the second slider convex portion 252q to the proximal end side A2 (the closing direction) than the corresponding main body convex portion (the main body first convex portion 251p, the main body second convex portion 252p), the pair of arms 21 transition to the swing form (the second form).

The swing form (the second form) is an operation form in which one of the first arm 211 and the second arm 212 may be opened and closed, and the opening operation of the other arm is regulated by the regulating member 250B. The first slider convex portion 251q that has moved to the proximal end regulates the movement of the first operating wire 231 to the distal end side A1 (the opening direction), thereby regulating the opening operation of the first arm 211. Alternatively, the second slider convex portion 252q that has moved to the proximal end regulates the movement of the second operating wire 232 to the distal end side A1 (the opening direction), thereby regulating the opening operation of the second arm 212. As shown as an exemplary example in FIG. 17, the operator regulates the movement of the second operating wire 232 to the distal end side A1 (the opening direction) with the second regulating member 252B. The operator moves the first slider 243 to the distal end side A1, and moves only the first operating wire 231 to the distal end side A1 (the opening direction). As a result, only the first arm 211 is in the open form.

The clip device 300B according to the embodiment can suitably grip the tissue T in a lumen. Even when the tissue T is treated by the tangential approach, the clip 2 may be swung so that the pair of arms 21 are disposed to face the front surface of the tissue T which is the treatment region. The clip device 300B may easily regulate the opening operation of one of the pair of arms 21 (the first arm 211 and the second arm 212) that operate independently.

The second embodiment of the present invention has been described above in detail with reference to the drawings, but the specific configuration is not limited to the embodiment, and design changes and the like are also included within the scope that does not deviate from the gist of the present invention. Furthermore, the components shown in the embodiments and modified examples may be appropriately combined to form a configuration.

### Modified Example

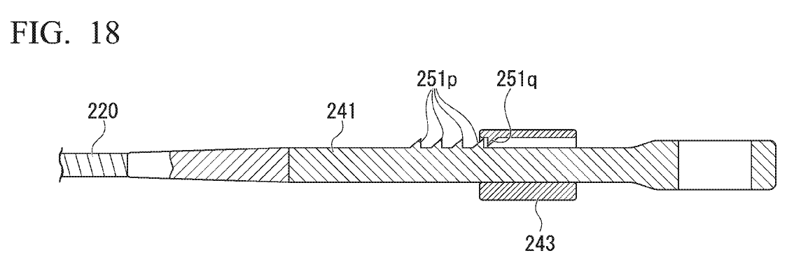

FIG. 18 shows a modified example of the regulating member 250B.

The operating part main body 241 may be provided with a plurality of main body first convex portions 251p. The plurality of main body first convex portions 251p are arranged along the longitudinal direction A. The degree of closure of the pair of arms 21 in the swing form (the second form) may be changed in stages.

### Third Embodiment

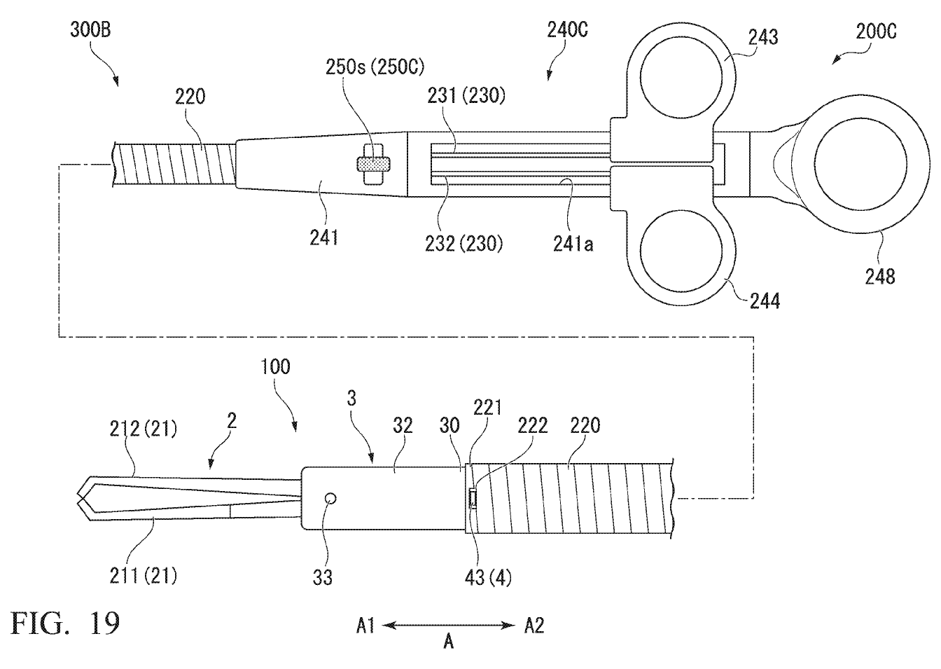

A clip device 300C according to a third embodiment of the present invention will be described with reference to FIGS. 19 to 22. In the following description, components that are common to those already described will be assigned the same reference numerals, and duplicate descriptions will be omitted.

FIG. 19 is a view showing a clip device 300C according to the embodiment.

The clip device (a clip system, an endoscopic treatment tool) 300C includes a clip unit 100 and an applicator 200C. The clip unit 100 is loaded into the applicator 200C that may be inserted into a channel of an endoscope, and is introduced into a treatment position.

The applicator (a clip unit introduction device) 200C includes a sheath 220, an operating wire 230, an operating part 240C, and a regulating member 250C.

The operating part 240C includes an operating part main body 241, a first slider 243, a second slider 244, and a thumb ring 248.

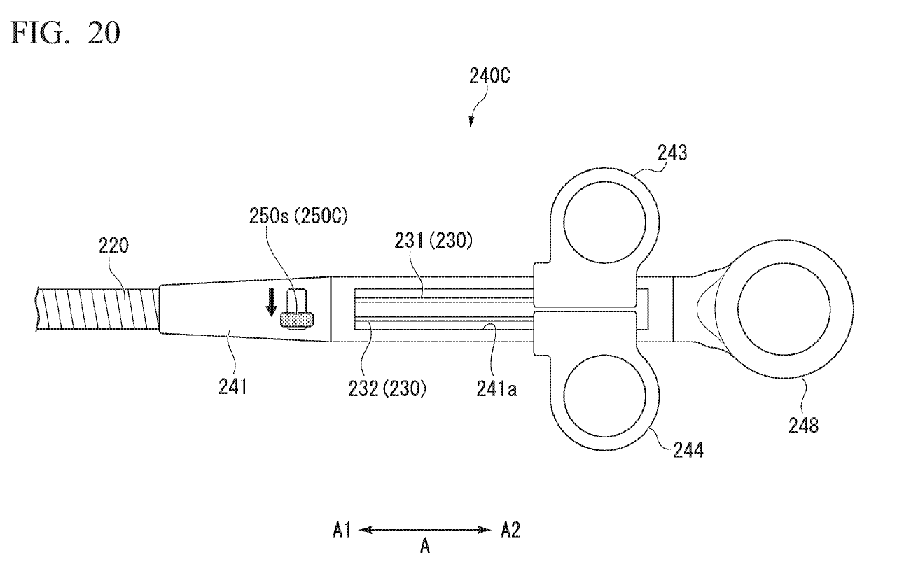

FIG. 20 is a view showing the regulating member 250C.

The regulating member 250C transitions the pair of arms 21 between the normal form (the first form) and the swing form (the second form).

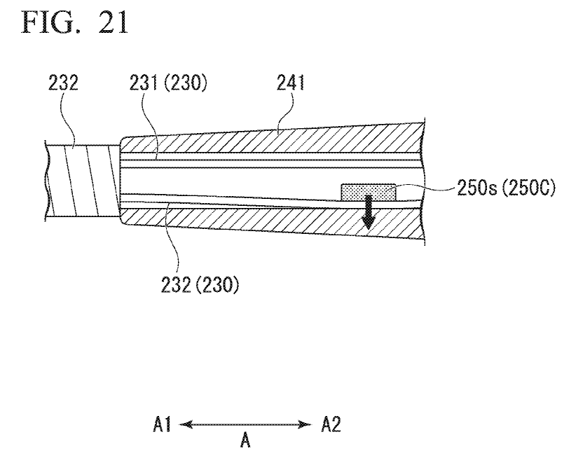

FIG. 21 shows an operation of the regulating member 250C.

The regulating member 250C is a wire stopper that regulates the movement of one of the first operating wire 231 and the second operating wire 232. The regulating member 250C is attached to the operating part main body 241 and has a slide member 250s that is slidable in an opposing direction in which the first operating wire 231 and the second operating wire 232 face each other. The slide member 250s in contact with the first operating wire 231 regulates the advance and retract movement of the first operating wire 231 in the longitudinal direction A. The slide member 250s in contact with the second operating wire 232 regulates the advance and retract movement of the second operating wire 232 in the longitudinal direction A.

By moving the slide member 250s to a position at which the slide member 250s does not come into contact with either the first operating wire 231 or the second operating wire 232, the pair of arms 21 transitions to the normal form (the first form).

The normal form (the first form) is an operation form in which the first arm 211 and the second arm 212 may be opened and closed. In the embodiment, the first arm 211 and the second arm 212 may be opened and closed independently. At this time, the slide member 250s does not regulate the advance and retract movement of the first operating wire 231 and the second operating wire 232.

By moving the slide member 250s to the position where the slide member 250s comes into contact with one of the first operating wire 231 and the second operating wire 232, the pair of arms 21 transition to the swing form (the second form).

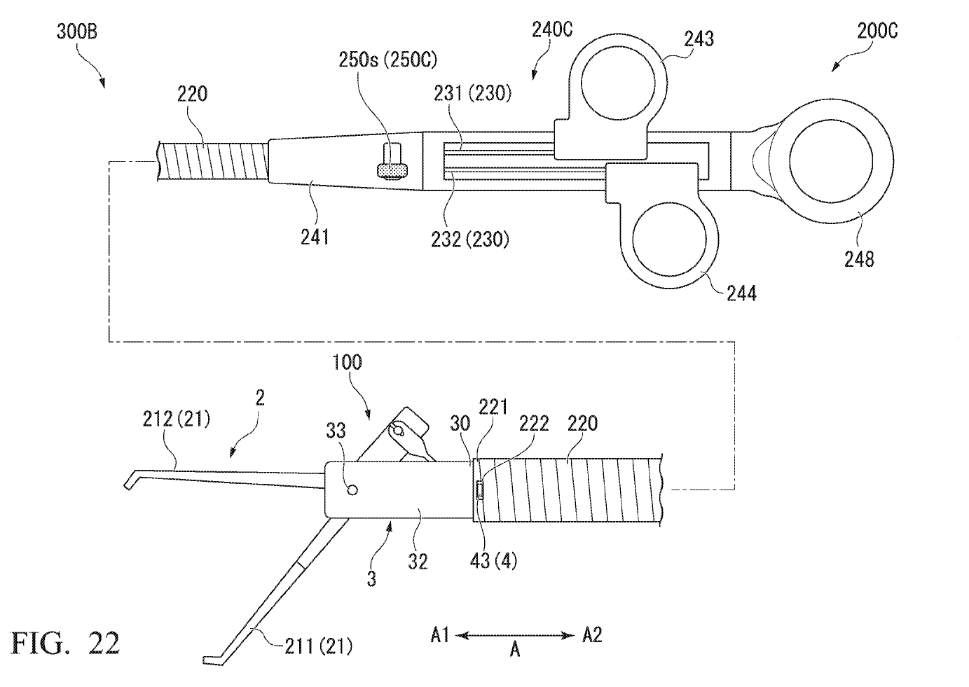

FIG. 22 shows the pair of arms 21 in the second form.

The swing form (the second form) is an operation form in which one of the first arm 211 and the second arm 212 may be opened and closed, and the opening operation of the other arm is regulated by the regulating member 250C. The slide member 250s in contact with the first operating wire 231 regulates the movement of the first operating wire 231, thereby regulating the opening operation of the first arm 211. Alternatively, the slide member 250s in contact with the second operating wire 232 regulates the movement of the second operating wire 232, thereby regulating the opening operation of the second arm 212. As shown as an exemplary example in FIG. 22, the operator regulates the movement of the second operating wire 232 to the distal end side A1 (the opening direction) with the regulating member 250C. The operator moves the first slider 243 to the distal end side A1, and moves only the first operating wire 231 to the distal end side A1 (the opening direction). As a result, only the first arm 211 is in the open form.

The clip device 300C according to the embodiment can suitably grip the tissue T in a lumen. Even when the tissue T is treated by the tangential approach, the clip 2 may be swung so that the pair of arms 21 are disposed to face the front surface of the tissue T which is the treatment region. The clip device 300C may easily regulate the opening operation of one of the pair of arms 21 (the first arm 211 and the second arm 212) that operate independently.

Although the third embodiment of the present invention has been described above in detail with reference to the drawings, the specific configuration is not limited to the embodiment, and design changes and the like are also included within the scope that does not deviate from the gist of the present invention. Furthermore, the components shown in the embodiment and modified example can be appropriately combined to form a configuration.

### Modified Example

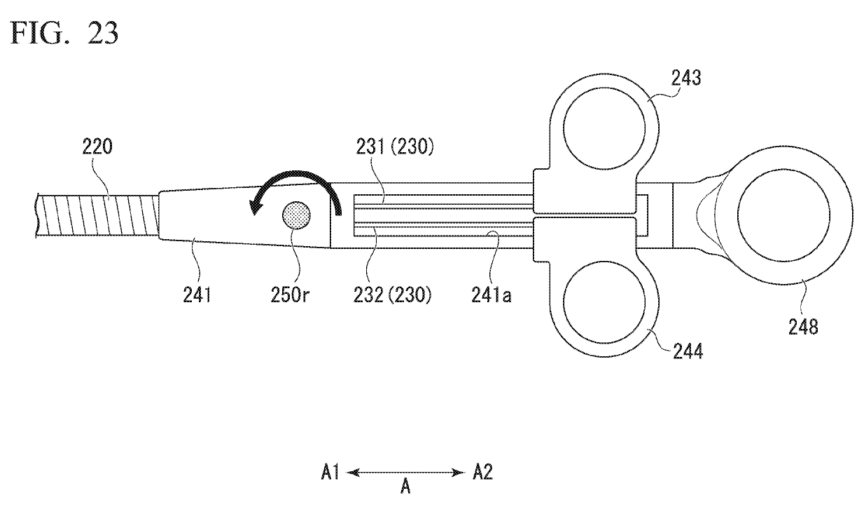

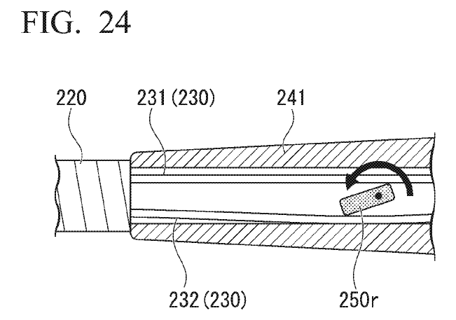

FIGS. 23 and 24 are views showing a rotating member 250r which is a modified example of the sliding member 250s. The rotating member 250r is rotatably attached to the operating part main body 241. By rotating, the rotating member 250r may be moved to a position at which the rotating member 250r does not come into contact with both the first operating wire 231 and the second operating wire 232, and may be moved to a position at which it comes into contact with one of the first operating wire 231 and the second operating wire 232.

### Modified Example

In the above embodiment, the regulating member 250 and the like may regulate the opening operation of the first arm 211 and the second arm 212. However, the regulating member 250 and the like may be configured to regulate the opening operation of only one of the first arm 211 and the second arm 212.

### Fourth embodiment

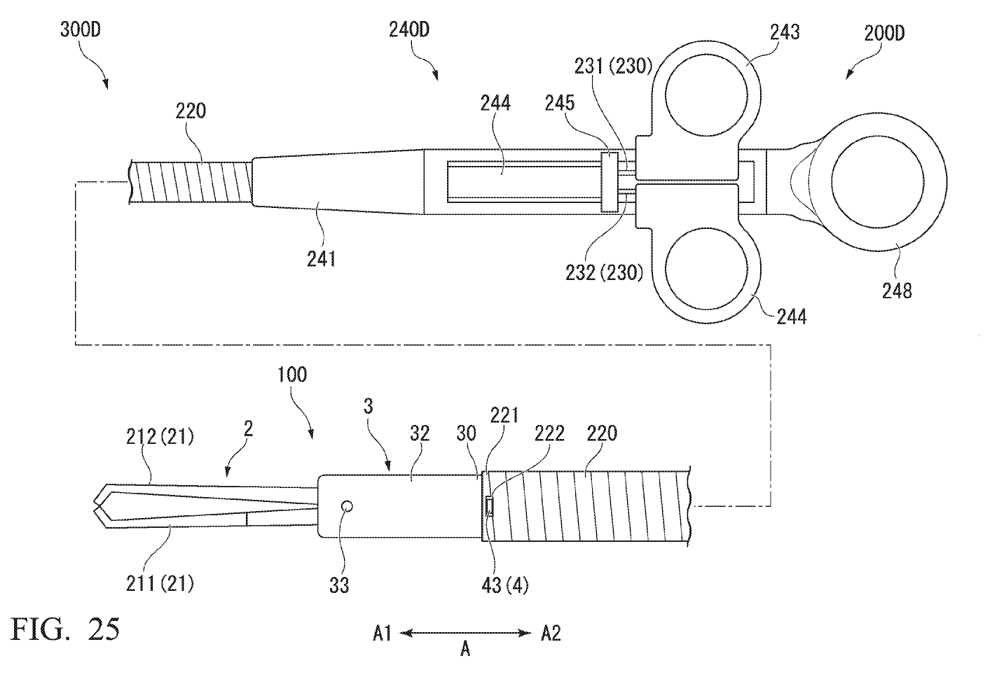

A clip device 300D according to a fourth embodiment of the present invention will be described with reference to FIGS. 25 to 27. In the following description, components that are common to those already described will be assigned the same reference numerals, and duplicate descriptions will be omitted.

FIG. 25 is a view showing the clip device 300D according to the embodiment.

The clip device (a clip system, an endoscopic treatment tool) 300D includes a clip unit 100 and an applicator 200D. The clip unit 100 is loaded into the applicator 200D that may be inserted through a channel of an endoscope, and is introduced into a treatment position.

The applicator (a clip unit introduction device) 200D includes a sheath 220, an inner sheath 224, an operating wire 230, an operating part 240D, and a regulating member 250D.

The inner sheath 224 is a cylindrical member that is inserted to be advanceable and retractable through an internal space of the sheath 220. The first operating wire 231 and the second operating wire 232 are disposed parallel to each other in an internal space 224s of the inner sheath 224.

The operating part 240D includes an operating part main body 241, a first slider 243, a second slider 244, an inner sheath slider 245, and a thumb ring 248.

The inner sheath slider 245 is attached to the operating part main body 241 to be advanceable and retractable in the longitudinal direction A, and a proximal end of the inner sheath 224 is attached thereto.

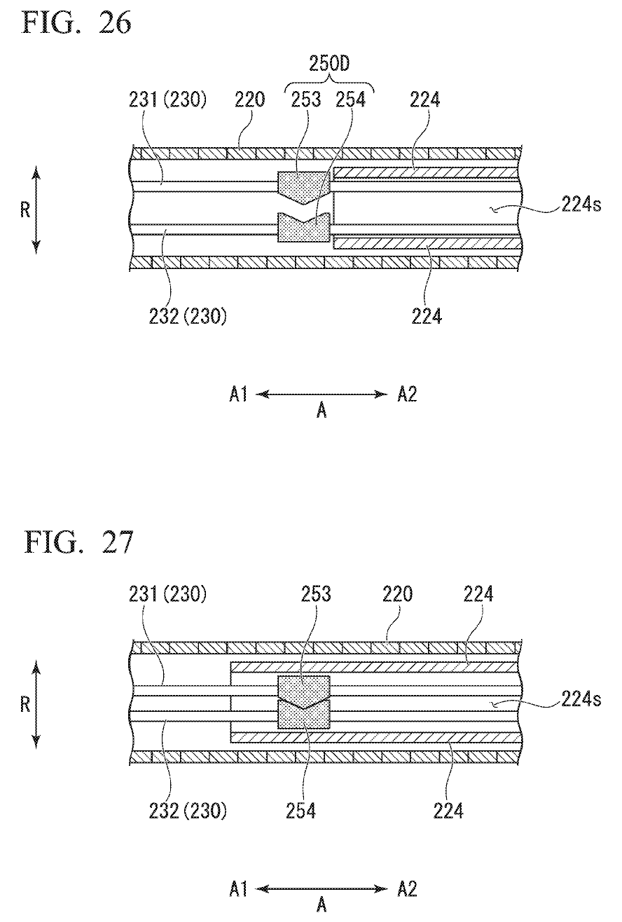

FIGS. 26 and 27 are views showing a regulating member 250D.

The regulating member 250D transitions the pair of arms 21 between an independent form (a third form) and a cooperative form (a fourth form).

The regulating member 250D regulates a relative operation of the first operating wire 231 and the second operating wire 232 in the longitudinal direction A, thereby causing the first operating wire 231 and the second operating wire 232 to perform a coordinative operation. The regulating member 250D has a first engaging portion 253 attached to the first operating wire 231 and a second engaging portion 254 attached to the second operating wire 232. As shown in FIG. 27, when the first engaging portion 253 and the second engaging portion 254, which are close to each other in a radial direction R, engage with each other, the relative operation of the first operating wire 231 and the second operating wire 232 in the longitudinal direction A is regulated.

When the first engaging portion 253 and the second engaging portion 254 are engaged with each other, it is desirable that an angle of the first arm 211 and an angle of the second arm 212 with respect to the longitudinal axis direction L of the sheath 220 be approximately the same.

As shown in FIG. 26, by moving the inner sheath slider 245 to the proximal end side A2 and moving the inner sheath 224 to a position at which the first engaging portion 253 and the second engaging portion 254 are not included in the internal space 224s, the pair of arms 21 transition to the independent form (the third form).

The independent form (the third form) is an operation form in which the first arm 211 and the second arm 212 may be opened and closed independently. In this case, the regulating member 250D does not regulate the relative operation of the first operating wire 231 and the second operating wire 232. In the independent form (the third form), for example, by further moving the first slider 243 (the first operating wire 231) to the distal end side A1 than the second slider 244 (the second operating wire 232), the pair of arms 21 may transition to a quasi-swing form (a quasi-second form) such as the swing form (the second form). The quasi-swing form (the quasi-second form) is an operation form in which one of the first arm 211 and the second arm 212 may be opened and closed, and the opening operation of the other arm is regulated by the user.

As shown in FIG. 27, by moving the inner sheath slider 245 to the distal end side A1 and moving the inner sheath 224 to the position at which the first engaging portion 253 and the second engaging portion 254 are included in the internal space 224s, the pair of arms 21 transition to the cooperative form (the fourth form).

The cooperative form (the fourth form) is an operation form in which the first arm 211 and the second arm 212 may be opened and closed in cooperation with each other. The first engaging portion 253 and the second engaging portion 254 which have moved into the internal space 224s of the inner sheath 224 engage with each other in the radial direction R. When the first engaging portion 253 and the second engaging portion 254 are engaged with each other, the relative operation of the first operating wire 231 and the second operating wire 232 are regulated.

The clip device 300D according to the embodiment can suitably grip the tissue T in a lumen. The clip device 300D can easily switch between the independent form (the third form) in which the first arm 211 and the second arm 212 can be opened and closed independently, and the cooperative form (the fourth form) in which the first arm 211 and the second arm 212 can be opened and closed in cooperation with each other, thereby suitably gripping the tissue T.

Although the fourth embodiment of the present invention has been described above in detail with reference to the drawings, the specific configuration is not limited to the embodiment, and design changes and the like are also included within the scope that does not deviate from the gist of the present invention. Furthermore, the components shown in the embodiments and modified examples can be appropriately combined to form a configuration.

### Modified Example

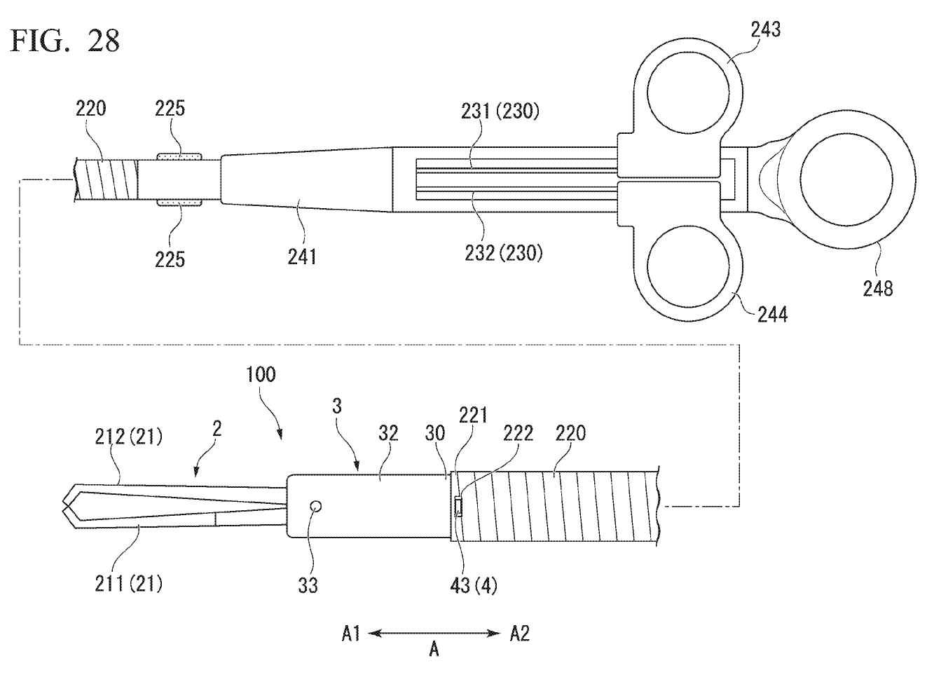

FIG. 28 shows a pressing member 225 which is a modified example of the inner sheath 224.

The pressing member 225 is an elastic member provided at the proximal end of the sheath 220. The pressing member 225 may be elastically deformed to protrude into the internal space of the sheath 220.

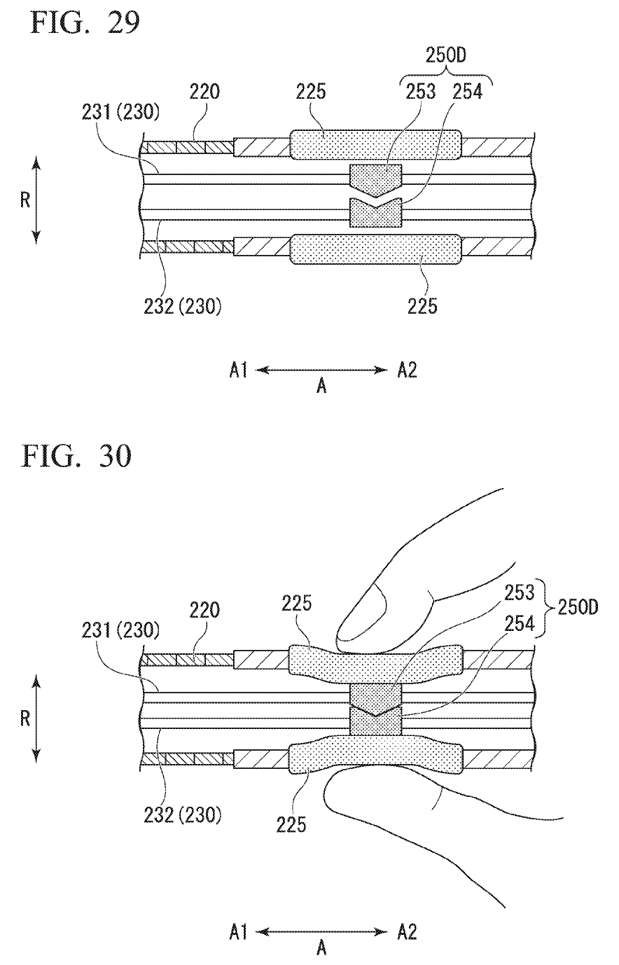

FIGS. 29 and 30 are views showing an operation of the pressing member 225.

The first engaging portion 253 and the second engaging portion 254 that are pressed from both sides in the radial direction R by the pressing member 225 engage with each other, thereby regulating the relative operation of the first operating wire 231 and the second operating wire 232.

### Modified Example

The pressing member 225 may be a hard member instead of an elastically deformable elastic member as shown in FIG. 30. For example, the pressing member 225, which is a hard member, is movable in the radial direction R with respect to the operating part main body 241 like a push switch. The pressing member 225, which is a hard member, is movable between a first position at which it is far away from the first engaging portion 253 and the second engaging portion 254, and a second position at which it is in contact with the first engaging portion 253 and the second engaging portion 254. By further having a structure that maintains a state in which the pressing member 225, which is a hard member, is disposed at the second position, the pressing member 225, which is a hard member, may maintain the engagement between the first engaging portion 253 and the second engaging portion 254 even when the user releases his/her hand from the pressing member 225.

### Fifth embodiment

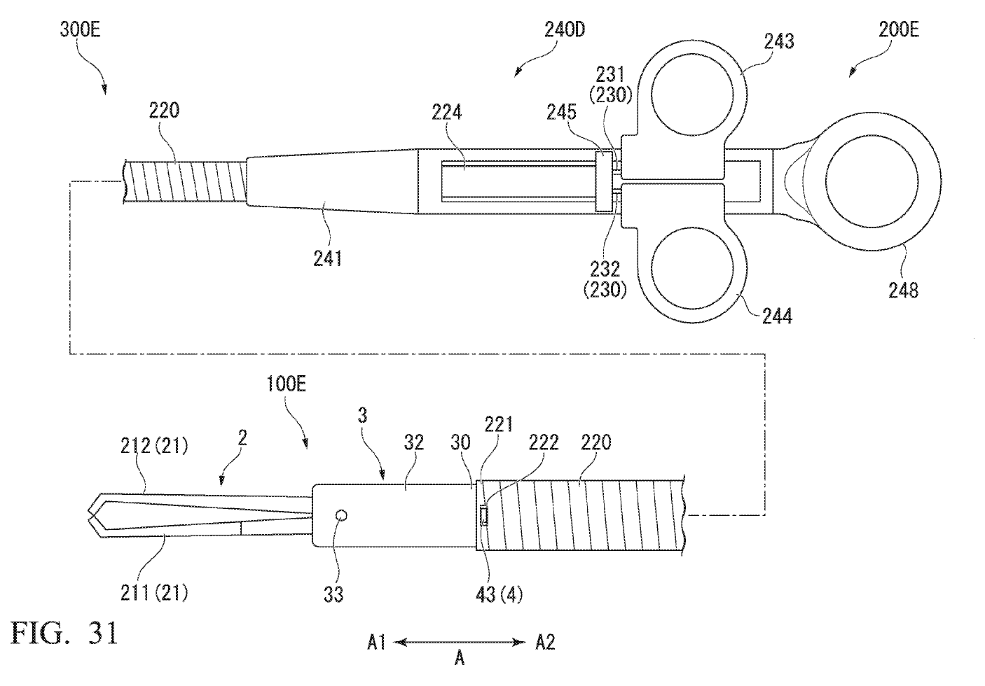

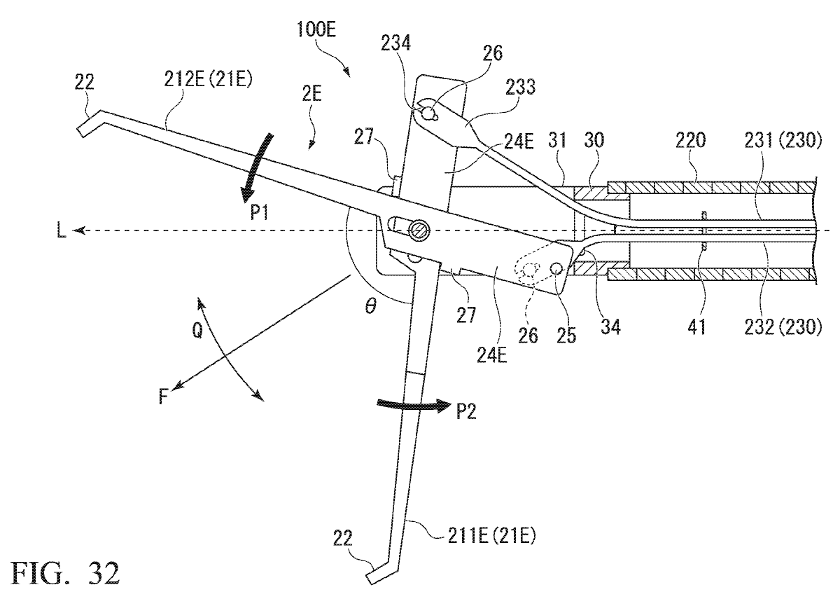

A clip device 300E according to a fifth embodiment of the present invention will be described with reference to FIGS. 31 and 32. In the following description, components that are common to those already described will be assigned the same reference numerals, and duplicate descriptions will be omitted.

FIG. 31 shows a clip device 300E according to the embodiment.

The clip device (a clip system, an endoscopic treatment tool) 300E includes a clip unit 100E and an applicator 200D. The clip unit 100E is loaded into the applicator 200D that may be inserted through a channel of an endoscope, and is introduced into a treatment position.

FIG. 32 shows the clip unit 100E.

The clip unit 100E is loaded into the distal end portion 221 of the sheath 220 of the applicator 200D. The clip unit 100E includes a clip 2E, a holder 3, and a connecting member 4.

The clip (a clip arm) 2E has a pair of arms 21E. The clip (the clip arm) 2E may have an auxiliary arm or the like other than the pair of arms 21E.

The pair of arms 21E may be opened and closed in the forward direction F, and may transition between the open form (the open state) and the closed form (the closed state). The pair of arms 21E may swing in the swing direction Q. The forward direction F of the pair of arms 21E changes as the pair of arms 21E swing.

The pair of arms 21E includes a first arm 211E and a second arm 212E. The first arm 211E and the second arm 212E are supported by the holder 3 to be rotatable in the opening-closing direction P around the rotation axis RO along the up-down direction B that is substantially perpendicular to the longitudinal direction A. The first arm 211E and the second arm 212E are supported separately by the holder 3.

In the opening-closing direction P, the direction in which one arm approaches the other arm is referred to as the “closing direction P1.” In the opening-closing direction P, the direction in which one arm moves away from the other arm is referred to as the “opening direction P2.” The direction in which the pair of arms 21E face each other is referred to as the “inner side,” and the side opposite to the inner side is referred to as the “outer side.” The operation of the pair of arms 21E transitioning from the open form (the open state) to the closed form (the closed state) is referred to as the “closing operation.” The operation of the pair of arms 21E transitioning from the closed form (the closed state) to the open form (open state) is referred to as the “opening operation.”

Each of the first arm 211E and the second arm 212E has, from the distal end side A1 to the proximal end side A2, a claw 22, a gripping portion 23, a main body portion 24E, and an engaging portion 25. The main body portion 24E has a connecting portion 26 and a link portion 27.

The link portion 27 engages with the other arm of the pair of arms 21E when the pair of arms 21E are in the swing form which will be described below. As shown in FIG. 32, the link portion 27 is provided at an end portion of the main body portion 24 in a width direction C that is substantially perpendicular to the longitudinal direction A and the up-down direction B. The link portion 27 extends inward from the main body portion 24. The claw 22 and the link portion 27 are provided on opposite sides of the rotation axis RO that face each other.

The pair of arms 21E may transition between the normal form (the third form) shown in FIG. 31 and the swing form (the fifth form) shown in FIG. 31.

The normal form (the third form) is an operation form in which the first arm 211E and the second arm 212E rotate independently. In this case, the regulating member 250D does not regulate the relative operation of the first operating wire 231 and the second operating wire 232.

The swing form (the fifth form) is an operation form in which one arm (one of the first arm 211E and the second arm 212E) that rotates in the opening direction P2 of the opening-closing direction P rotates the other arm (the other of the first arm 211E and the second arm 212E) in the closing direction P1 of the opening-closing direction P. When the pair of arms 21E are in the swing form, the forward direction F of the pair of arms 21E is along a direction intersecting the longitudinal axis direction L of the sheath 220, as shown in FIG. 32.

An angle at which the first arm 211E and the second arm 212E open in the forward direction F is defined as an opening-closing angle θ. When the opening-closing angle θ is less than a predetermined angle, the pair of arms 21E are in the normal form (the third form). When the opening-closing angle θ is the predetermined angle, the pair of arms 21E are in the swing form (the fifth form).

When the opening-closing angle θ reaches the predetermined angle, the link portion 27 of the first arm 211E, which rotates in the opening direction P2, engages with the second arm 212E. Also, the first arm 211E engages with the link portion 27 of the second arm 212E. When the first arm 211E further rotates in the opening direction P2, the link portion 27 of the first arm 211E rotates the second arm 212E in the closing direction P1 of the opening-closing direction P. The first arm 211E also rotates the link portion 27 of the second arm 212E in the closing direction P1 of the opening-closing direction P. As a result, the pair of arms 21E swing in the swing direction Q. The swing direction (a rotation direction) Q substantially coincides with the opening-closing direction P of the pair of arms 21E. In other words, a plane on which the pair of arms 21E swing (a plane through which the pair of swinging arms 21E pass) and a plane that is opened and closed by the pair of arms 21E (a plane through which the pair of opening and closing arms 21E pass) approximately coincide with each other.

When the opening-closing angle θ is a predetermined angle, the second arm 212E rotating in the opening direction P2 of the opening-closing direction P similarly rotates the first arm 211E in the closing direction P1 of the opening-closing direction P. As a result, the pair of arms 21E swing in the swing direction Q.

For example, when the first arm 211E that has been rotating in the opening direction P2 rotates in the closing direction P1, the opening-closing angle θ becomes less than the predetermined angle. When the opening-closing angle θ becomes less than the predetermined angle, the pair of arms 21E returns to the normal form (the third form).

As in the fourth embodiment, by operating the inner sheath slider 245, the regulating member 250D transitions the pair of arms 21E between the independent form (the third form) and the cooperative form (the fourth form).

The clip device 300E according to the embodiment can suitably grip the tissue T in a lumen. The clip device 300E can easily switch between the independent form (the third form) in which the first arm 211E and the second arm 212E can be opened and closed independently, the cooperative form (the fourth form) in which the first arm 211E and the second arm 212E can be opened and closed in coordination with each other, and the swing form (the fifth form) to suitably grip the tissue T.

Although the fifth embodiment of the present invention has been described above in detail with reference to the drawings, the specific configuration is not limited to the embodiment, and design changes and the like are also included within the scope that does not deviate from the gist of the present invention. Furthermore, the components shown in the embodiments and modified examples can be appropriately combined to form a configuration.

What is claimed is:

1. A clip device comprising:
a clip unit including arms having a first arm and a second arm, and a holder configured to support the arms;
a sheath separably connected to the clip unit; and
a regulating member configured to regulate an opening operation in which the first arm transitions from a closed form to an open form,
wherein the arms are transitioned between a first form in which the first arm and the second arm are openable and closeable, and a second form in which the second arm is openable and closeable and the opening operation of the first arm is regulated by the regulating member.

2. The clip device of claim 1, further comprising a second regulating member configured to regulate an opening operation in which the second arm transitions from the closed form to the open form.

3. The clip device of claim 1, wherein when the arms are in the first form, the first arm and the second arm are openable and closeable in coordination with each other.

4. The clip device of claim 1, wherein when the arms are in the first form, the first arm and the second arm are openable and closeable independently.

5. The clip device of claim 1, further comprising:
a first wire that passes through the sheath and is separably connected to the first arm; and
a second wire that passes through the sheath and is separably connected to the second arm,
wherein the first wire is configured to move in an opening direction to cause the first arm to perform the opening operation, and
the regulating member regulates movement of the first wire in the opening direction to regulate the opening operation of the first arm.

6. The clip device of claim 5, further comprising a slider connected to the first wire and the second wire,
wherein the regulating member includes a mechanism that regulates the movement of the first wire in the opening direction regardless of an operation of the slider that moves the first wire in the opening direction.

7. The clip device of claim 5, further comprising:
a first slider connected to the first wire; and
a second slider connected to the second wire,
wherein the regulating member includes a ratchet that regulates an operation of the first slider that moves the first wire in the opening direction.

8. The clip device of claim 5, further comprising:
a first slider connected to the first wire; and
a second slider connected to the second wire,
wherein the regulating member includes a wire stopper that regulates the movement of the first wire.

9. The clip device of claim 6, further comprising an operating part to which the sheath is attached,
wherein the regulating member is provided at the operating part.

10. A clip device comprising:
a clip unit including arms having a first arm and a second arm, and a holder configured to support the arms;
a sheath separably connected to the clip unit; and
a regulating member configured to regulate the first arm and the second arm to operate in cooperation with each other,
wherein the arms are transitioned between a third form in which the first arm and the second arm are openable and closeable independently, and a fourth form in which the first arm and the second arm are openable and closeable in coordination with each other due to the regulating member.

11. The clip device of claim 10, further comprising:
a first wire that passes through the sheath and is separably connected to the first arm; and
a second wire that passes through the sheath and is separably connected to the second arm;
wherein the regulating member regulates a relative operation between the first wire and the second wire, thereby causing the first arm and the second arm to operate in coordination with each other.

12. The clip device of claim 11, further comprising:
a first slider connected to the first wire; and
a second slider connected to the second wire;
wherein the regulating member includes a first engaging portion attached to the first wire and a second engaging portion attached to the second wire, and
when the first engaging portion and the second engaging portion engage with each other, the relative operation between the first wire and the second wire is regulated.

13. The clip device of claim 12, further comprising an inner sheath through which the sheath is inserted to be advanceable and retractable,
wherein the first wire and the second wire are inserted through the inner sheath, and
when the inner sheath is disposed at a position at which the first engaging portion and the second engaging portion are inserted thereinto, the first engaging portion and the second engaging portion engage with each other.

14. The clip device of claim 10, wherein the arms are further transitioned to a fifth form in which the first arm that rotates in an opening direction rotates the second arm in a closing direction.

15. The clip device of claim 14, wherein an angle at which the first arm and the second arm open is defined as an opening-closing angle, and the arms are configured to transition from the third form to the fifth form when the opening-closing angle widens to a predetermined angle.

16. The clip device of claim 15, wherein the first arm has a link portion that engages with the second arm when the arms are in the fifth form.

17. The clip device of claim 16, wherein the first arm is supported by the holder to be rotatable around a rotation axis, and
a distal end of the first arm and the link portion of the first arm are provided on opposite sides of the rotation axis.

18. The clip device of claim 11, further comprising a connecting member configured to separably connect the sheath and the holder,
wherein the connecting member is configured to separate the sheath and the holder when the first wire and the second wire are pulled.

19. The clip device of claim 18, wherein the clip unit has a locking mechanism that is configured to lock the arms in a closed form.

20. The clip device of claim 19, wherein the locking mechanism has an engaged portion provided on the holder and an engaging portion provided on the arms, and
the arms are configured to be locked in the closed form when the arms are pulled by the first wire and the second wire and the engaging portion engages with the engaged portion.
